(19) United States
(12) Patent Application Publication
(10) Pub. No.: US 2020/0262903 A1
(43) Pub. Date: Aug. 20, 2020

(54) Treatment of age-related macular degeneration

(71) Applicant: Henry J. Smith, Temecula, CA (US)

(72) Inventor: Henry J. Smith, Temecula, CA (US)

(21) Appl. No.: 16/501,058

(22) Filed: Feb. 15, 2019

(51) Int. Cl. C07K 16/22; A61K 9/107; A61K 9/00; A61K 47/69

(52) U.S. Cl. CPC C07K 16/22; A61K 9/107; A61K 45/06; A61K 47/6907; A61K 47/6913; A61K 9/0048

(57) ABSTRACT

A means of treating both the “dry” and/or “wet” forms of Age-Related Macular Degeneration (ARMD) using a disease targeting drug delivery system in which an anti-inflammatory drug is incorporated into nanoparticles such as liposomes, micelles, dendrimers, lipid nanospheres, nanoemulsions and the like. The nanoparticles are coated with an anti-Vascular Endothelial Growth Factor Receptor (VEGFR) targeting agent such as anti-VEGFR antibodies, anti-VEGFR aptamers, anti-VEGFR binding peptides and the like. Upon administration into the eye of a patient with ARMD the targeting agent on the nanoparticle will bind to VEGFR on neovascular cells in the retina and inhibit the abnormal proliferation of new blood vessels. In addition to its therapeutic action the targeting agent by binding to its receptor will anchor the drug delivery vehicle at the site of inflammation where the anti-inflammatory drug is released for maximum effect in inhibiting the local inflammatory response.

### CROSS-REFERENCE TO RELATED APPLICATIONS

This patent application claims priority to provisional patent application No. 62/710,615 titled “Treatment of Age-Related Macular Degeneration” and filed Feb. 22, 2018.

### STATEMENT REGARDING FEDERALLY SPONSORED RESEARCH OR DEVELOPMENT

Not Applicable

### BACKGROUND TO THE INVENTION

There are about 2 million people in the United States living with some form of Age-Related Macular Degeneration (ARMD). ARMD is a major cause of blindness and visual impairment in older adults. Approximately 10% of adults 66 to 74 years of age have findings of macular degeneration. The prevalence increases to 30% in adults 75 to 85 years of age. There are two types of ARMD that are categorized as “dry” (atrophic) ARMD and “wet” (neovascular) ARMD. Dry ARMD affects approximately 90% of individuals with ARMD. The more severe wet ARMD affects approximately 10% of individuals with ARMD but accounts for about 90% of severe vision loss from the disease. All people who have the wet form had the intermediate stage of the dry form first. The dry form can also suddenly turn into the wet form, even during early stage ARMD.

In dry (atrophic) ARMD cellular debris called drusen accumulates between the retina and the choroid. It has been suggested that the accumulation of drusen is related to some form of inflammation and atherosclerotic plaque formation. In some individuals the accumulation of drusen appears to stimulate an abnormal proliferation of blood vessels in the choroid behind the retina leading to the retina becoming detached. This leads to the condition of the wet (neovascular) ARMD.

It appears that the dry and wet forms of ARMD represent the opposite ends of a disease spectrum that has both an inflammatory component and an abnormal vascular component. It would be desirable if an effective means of treating ARMD were developed that could treat both conditions.

This invention discloses a novel means of treating ARMD using immunoliposomes or similar nanosized drug delivery vehicles coated with an anti-Vascular Endothelial Growth Factor Receptor (VEGFR) targeting agent, and incorporating an anti-inflammatory drug. This compound biopharmaceutical has two modes of action; first the anti-VEGFR targeting agent on the exterior of the drug delivery vehicle will bind to neovascular cells in the retina and inhibit them from proliferating and forming abnormal blood vessels; and second the anti-inflammatory drug is released from the drug delivery vehicle in the vicinity of the retina and will inhibit the local inflammatory response.

The art is silent on a means of treating ARMD using anti-VEGFR targeting agents (e.g. antibodies, aptamers, binding peptides, and the like) attached to the exterior of nanosized drug delivery vehicles (e.g. liposomes, micelles, dendrimers, nanoemulsions, nanoparticles, and the like) incorporating an anti-inflammatory drug. In this invention the terms “drug delivery vehicle”, “nanocarrier” and “nanoparticles” will refer to any one of a variety of nanosized drug delivery systems including liposomes, micelles, dendrimers, nanoemulsions, nanoparticles, and the like.

### SUMMARY OF THE INVENTION

A means of treating both the “dry” and/or “wet” forms of Age-Related Macular Degeneration (ARMD) using a disease targeting drug delivery system in which an anti-inflammatory drug is incorporated into nanoparticles such as liposomes, micelles, dendrimers, lipid nanospheres, nanoemulsions and the like. The nanoparticles are coated with an anti-Vascular Endothelial Growth Factor Receptor (VEGFR) targeting agent such as anti-VEGFR antibodies, anti-VEGFR aptamers, anti-VEGFR binding peptides and the like. Upon administration into the eye of a patient with ARMD the targeting agent on the nanoparticle will bind to VEGFR on neovascular cells in the retina and inhibit the abnormal proliferation of new blood vessels. In addition to its therapeutic action the targeting agent by binding to its receptor will anchor the drug delivery vehicle at the site of inflammation where the anti-inflammatory drug is released for maximum effect in inhibiting the local inflammatory response.

### DETAILED DESCRIPTION OF THE INVENTION

This invention discloses a novel means for simultaneously treating both the vascular component and the inflammatory component associated with ARMD using a biopharmaceutical possessing a dual therapeutic action. Said biopharmaceutical comprises an anti-inflammatory drug incorporated into a nanosized drug delivery vehicle coated with an anti-VEGFR targeting agent. Upon administration of this biopharmaceutical into the eye of a patient with ARMD the drug delivery vehicles will concentrate within the retinal layer. The anti-VEGFR targeting agent will bind to the VEGFR on neovascular cells in the retina and inhibit the abnormal proliferation of the retinal blood vessels. At the same time the anti-inflammatory drug is released from the drug delivery vehicle and will inhibit the local inflammatory process.

The art is silent on the use of a VEGFR targeting agent that acts as a vascular therapeutic agent, and which at the same time functions as a component of a drug delivery vehicle to deliver an anti-inflammatory drug to the retinal layer of the eye.

Vascular endothelial growth factor (VEGF) is an important signaling protein involved in angiogenesis. Since the discovery of VEGF several different types of VEGF have been identified (i.e. VEGF-A, VEGF-B, VEGF-C, VEGF-D and VEGF-E), as well as several different VEGF receptors (i.e. VEGFR-1/Flt-1, VEGFR-2/Flk-1/KDR and VEGFR-3/Flt-4) (Ferrara 2004; Hoeben et al. 2004). Of these VEGF-A appears to play a major role in angiogenesis by binding to VEGFR-2 and stimulating the vascular cells to form new blood vessels. There are also reports that Placental Growth Factor (PIGF) and VEGFR-1 may be involved in angiogenesis.

In one embodiment of this invention a means of preparing an anti-angiogenesis and anti-inflammation biopharmaceutical is described in which anti-VEGFR antibodies that can bind to VEGFR-1 and VEGFR-2 are attached to the exterior of liposomes, or nanoemulsions, or other nanocarriers incorporating an anti-inflammation drug.

In another embodiment of this invention a means of preparing an anti-angiogenesis and anti-inflammation biopharmaceutical is described in which specific anti-VEGFR-2 antibodies are attached to the exterior of liposomes, or nanoemulsions, or other nanocarriers incorporating an anti-inflammatory drug.

In another embodiment of this invention a means of preparing an anti-angiogenesis and anti-inflammation biopharmaceutical is described in which specific anti-VEGFR-1 antibodies are attached to the exterior of liposomes, or nanoemulsions, or other nanocarriers incorporating an anti-inflammatory drug.

Preparation of Anti-VEGFR Antibody.

VEGFR-1 and VEGFR-2 have a high degree of homology. It is possible to prepare antibodies that will bind to both receptors. However, each also has certain unique epitopes present that differentiate one from the other, and it is therefore also possible to prepare antibodies that are specific to either VEGFR-1 or to VEGFR-2. The procedures for developing anti-VEGFR-1 antibody and/or anti-VEGFR-2 antibody are essentially the same.

In this invention the term anti-VEGFR antibody will refer to either anti-VEGFR-1 antibody and/or to anti-VEGFR-2 antibody and/or to an antibody that binds to both VEGFR1/VEGFR2. The term antibody will include the whole antibody molecule; and/or the binding fragments Fab and Fab2 of the molecule; and/or in the case of a recombinant antibody either the whole recombinant molecule or the VEGFR binding fragment of the molecule, or the VEGFR binding fragment when it is a part of a fusion protein.

The anti-VEGFR antibody can be prepared as a polyclonal antibody in immunized animals, or it may be a monoclonal antibody prepared using hybridoma technology in mice or other animals. The monoclonal antibody may be “humanized” using genetic engineering methods. It may also be prepared as a recombinant fully human antibody protein using phage display technology. These and other methods of preparing antibodies using hybridoma technology or genetic engineering technology are well-known to those of skill in the art and are included within the scope of this invention.

There are many different means of preparing antibodies that are well-known to those of skill in the art. These are described and referenced in the teaching of Zhu (2011). This teaching is hereby included within the scope of this invention.

Drug Delivery Vehicle

In one embodiment of this invention the drug delivery vehicle is a liposome. Liposomes are nanosized lipid vesicles. Some liposomes are composed of a single bilayer lipid membrane enclosing an aqueous interior, while others are composed of multiple concentric lipid membranes separated by liquid layers. In the preferred embodiment of this invention the liposomes are composed of a single lipid bilayer and manufactured to be of a standard size. The diameter of the liposome is selected to be a uniform size between 20 nm and 250 nm, preferably between 50 nm and 150 nm and most preferably to be about 100 nm.

The liposomes can be prepared using a mixture of one or more of the following compounds: egg phosphatidylcholine (EPC), soy phosphatidylcholine (SPC), hydrogenated soy phosphatidylcholine (HSPC), phosphatidylethanolamine (PE), phosphatidylglycerol (PG), phosphatidylinsitol (PI), monosialoganglioside and sphingomyelin (SPM); the derivatized vesicle forming lipids such as poly(ethylene glycol)-derivatized distearoylphosphatidylethanolamine (DSPE-PEG), poly(ethylene glycol)-derivatized ceramides (CER-PEG), distearoylphosphatidylcholine (DSPC), dimyristoyl-phosphatidylcholine (DMPC), dimyristoylphosphatidylglycerol (DMPG), and dipalmitoylphosphatidylcholine (DPPC), and cholesterol.

In this invention a quantity of 1,2-Distearoyl-sn-Glycero-3-Phosphoethanolamine-N-PDP [PolyethyleneGlycol 2000] (DSPE-PEG 2000) and 1,2-Distearoyl-sn-Glycero-3phosphoethanolamine-N-Maleimide [Polyethylene Glycol 2000] (DSPE-PEG 2000-Maleimide) is added to the lipid mixture. The DSPE-PEG 2000 will incorporate into the lipid layer of liposome with the PEG polymer extending out into the exterior medium. The presence of the PEG polymer attached to the exterior surface of the liposomes appears to stabilize and prevent them from aggregating. Also anchored to the liposome membrane is DSPE-PEG 2000-maleimide, with the maleimide group being free and able to be chemically coupled to the Fab fragment of the anti-VEGFR antibody. Liposomes that have an antibody or the binding fragment of an antibody attached to their exterior surface are termed “immunoliposomes”.

In one embodiment of this invention the drug delivery vehicle is a nanoemulsion. Nanoemulsions are comprised of oil-in-water nanodroplets in which a lipid soluble anti-inflammatory drug is dissolved in the oil/lipid component. The diameter of the nanodroplet is manufactured to be a uniform size between 20 nm and 250 nm, preferably between 50 nm and 150 nm, and most preferably to be about 100 nm.

It is obvious to those of skill in the art that other nanosized drug delivery vehicles such as micelles, dendrimers, nanocapsules, lipid nanoparticles and the like can be used in lieu of liposomes and nanoemulsions. The procedures for manufacturing said nanocarriers are well known to those of skill in the art. Such methods are included by reference and are within the scope of this invention.

A wide variety of anti-inflammatory drugs can be enclosed or incorporated into the various types of nanocarriers. For example, water soluble drugs can be encapsulated within the aqueous center of an immunoliposome or loaded using a gradient technique. Lipid soluble drugs can be co-dissolved with the lipid mixture and thus become incorporated into the lipid membrane of the immunoliposome, or they can be dissolved in a mixture of oil and lipid and incorporated into a nanoemulsion or lipid based nanoparticle.

In this invention the term “anti-inflammatory drug” is used to described any compound that either directly or indirectly inhibits the inflammatory process. This includes corticosteroids such as cortisone, hydrocortisone, prednisone, prednisolone, methylprednisolone, triamcinolone, triamcinolone acetonide, dexamethasone, betamethasone and their analogs. Also included under the term anti-inflammation drugs are immune inhibiting drugs such as methotrexate and immune modulating drugs such as cyclosporine, rapamycin, prograf and the like.

Materials and Methods:

In one embodiment of this invention the VEGFR targeting biopharmaceutical is prepared using liposomes as the nanocarrier of the anti-inflammatory drug. The following is an example of the preparation of an immunoliposomal formulation of water soluble glucocorticoids such as triamcinolone acetonide phosphate and prednisolone phosphate. First a mixture of SPC, cholesterol, DSPE-PEG 2000, and DSPE-PEG 2000-malemide are dissolved in an organic solvent such as chloroform:methanol. The lipid solution is then dried under vacuum and mild heating until a lipid film is formed. The lipid film is then hydrated using a glucocorticoid such as prednisolone phosphate in solution and homogenized to prepare multilamella liposomes encapsulating the drug within the aqueous interior of the liposome. The liposomes are then sonicated to prepare unilamella liposomes which are then downsized using a commercial homogenizer/extruder by successive extrusions through membranes of decreasing pore sizes. Final extrusion through a membrane of 80 nm pore size results in the production of uniform unilamella liposomes with a mean diameter of about 100 nm. The temperature of the constituents throughout this procedure is maintained at an elevated temperature that is set above the transition temperature of the lipid mixture.

The unilamella liposomes thus formed are composed of a lipid bilayer membrane encapsulating the drug. Liposomes thus prepared have anchored in the lipid layer the DSPE-PEG molecule with the distal end of the PEG chain being free. Also anchored in the lipid layer is the DSPE-PEG maleimide molecule with the distal end of the PEG chain bearing the active maleimide group being free. To attach the purified anti-VEGFR antibody to the liposome the antibody molecule is first treated to prepare the Fab fragment which is then chemically conjugated to the maleimide group.

The Fab fragment is prepared by treating the anti-VEGFR antibody with an immobilized preparation of the enzyme papain in the presence of excess cysteine. The Fc fragment and any remaining whole antibodies are removed by binding to immobilized Protein A. The Fab fragments are then incubated with the liposomes where conjugation of the Fab fragment to the DSPE-PEG-Maleimide chain will occur through an amide linkage. The Fab bound liposomes thus formed are separated from unbound material by dialysis or passage through a Sephadex column. The drug immunoliposomes thus prepared are stored at 4 C.

Alternatively, the drug liposomes are prepared with the DSPE-PEG-maleimide component omitted from the original lipid mixture. Instead the Fab antibody fragment is separately conjugated to the DSPE-PEG-maleimide molecule and the conjugated compound is then incubated with the drug liposomes at an elevated temperature above the transition temperature of the lipids resulting in the insertion of the DSPE component of the DSPE-PEG-Fab molecule into the lipid bilayer of the liposome. The Fab bound liposomes thus formed are separated from unbound material by dialysis or passage through a Sephadex column. The drug immunoliposomes thus prepared are stored at 4 C until used.

The immunoliposomes are thus prepared to have a uniform mean diameter that will be within the range of 20 nm to 250 nm; preferably within the range of 50 nm to 150 nm; and more preferably to be about 100 nm.

In one embodiment of this invention the VEGFR targeting nanocarrier is prepared as a nanoemulsion incorporating a lipid soluble anti-inflammatory drug. The following is an example of the preparation of a nanoemulsion incorporating the lipid soluble drug triamcinolone acetonide. First a mixture of SPC, cholesterol, DSPE-PEG 2000, and DSPE-PEG 2000-malemide are dissolved in an organic solvent such as chloroform:methanol. Triamcinolone acetonide is separately dissolved in an oil such as coconut oil, or soybean oil, or another natural or synthetic oil and then added to the lipid solution. The oil/lipid solution is then placed under vacuum and mild heating to remove the organic solvent leaving an oily liquid behind. This is then mixed with heated distilled water containing a surfactant such as Tween 20 and homogenized to form a coarse oil-in-water emulsion. The emulsion is sonicated and downsized using a commercial homogenizer/extruder, by successive extrusions through membranes of decreasing pore sizes. Final extrusion through a membrane of 80 nm pore size results in the production of oil nanodroplets with a mean diameter of about 100 nm. The temperature of the constituents throughout this procedure is maintained at an elevated temperature that is set above the transition temperature of the oil/lipid mixture.

The nanodroplets thus formed are composed of an internal core of oil surrounded by a monolayer of lipid. Embedded within the lipid monolayer is the lipid component of the DSPE-PEG molecule with the distal end of the PEG chain being free. Also anchored in the lipid layer is the DSPE-PEG maleimide molecule with the distal end of the PEG chain bearing the active maleimide group being free. To attach the purified anti-VEGFR antibody to the nanodroplet the antibody molecule is first treated to prepare the Fab fragment which is then chemically conjugated to the maleimide group.

The Fab fragment is prepared by treating the anti-VEGFR antibody with an immobilized preparation of the enzyme papain in the presence of excess cysteine. The Fc fragment and any remaining whole antibodies are removed by binding to immobilized Protein A. The Fab fragments are then incubated with the nanoemulsion where conjugation of the Fab fragment to the DSPE-PEG-Maleimide chain will occur through an amide linkage. The Fab bound nanoemulsion thus formed are separated from unbound material by passage through a Sephadex column and stored at 4 C.

The nanodroplets comprising the nanoemulsion are manufactured to have a uniform mean diameter that will be within the range of 20 nm to 250 nm; preferably within the range of 50 nm to 150 nm; and more preferably to be about 100 nm.

The methods of preparing liposomes, nanoemulsions, micelles, dendrimers, nanocapsules and nanoparticles are well known in the art and are included by reference to be within the scope of this invention (Torchilin V. P. 2007, Jain K. K. 2005). The methods of attaching a targeting moiety to their surface are also well known in the art (Park J. W. et al. 1997; 2002; Vasir J. K. et al. 2005) and are included by reference to be within the scope of this invention. All references describing these methods are hereby incorporated and considered to lie within the scope of this invention

### Discussion

The current means of treating individuals with the dry form of Age-Related Macular Degeneration is to administer a therapeutic dosage of corticosteroid into the vitreous humor of the eye. For example, Ozurdex® (biodegradable dexamethasone implant developed by Allergan), Retisert® (non-biodegradable fluocinolone acetonide implant developed by Bausch & Lomb), and Trivaris® (triamcinolone acetonide suspension developed by Allergan).

Individuals with the wet form of Age-Related Macular Degeneration receive a different means of therapy aimed at inhibiting the abnormal proliferation of the retinal blood vessels. It is believed that these individuals have an excess of Vascular Endothelial Growth Factor (VEGF) present in the vitreous humor of the eye and this stimulates the neovascular cells in the retina to proliferate in an uncontrolled fashion. The current means of treatment for the wet form of ARMD utilizes an aptamer to VEGF (Macugen®) that blocks the VEGF from binding to the VEGFR on vascular cells and thereby inhibits them from proliferating. More recently an antibody to VEGF (Lucentis®) has been used to bind to VEGF and block it from binding to VEGFR on vascular cells, and thereby inhibit them from proliferating

In this invention we propose a novel means of treating ARMD using an antibody or antibody-like binding agent that does not target VEGF as disclosed by prior art, but instead targets VEGFR present on neovascular cells in the retinal layer. The art is silent on the use of anti-VEGFR antibody or other anti-VEGFR binding agents such as anti-VEGFR aptamer and anti-VEGFR binding peptide to treat ARMD.

Further, in this invention we propose using a nanosized drug delivery vehicle such as liposomes, or nanoemulsion or other nanocarriers to carry an anti-inflammatory drug to the site of inflammation within the eye. The nanocarriers are prepared so that the exterior surface of the nanocarrier is coated with an anti-VEGFR binding agent. The art is silent on the use of anti-VEGFR targeted nanocarriers incorporating an anti-inflammatory drug as a means of treating ARMD.

Huber et al. (1999) reported that anti-VEGFR-2 (Flk-1) antibody inhibits tumor angiogenesis; Luttun et al. (2002) reported that anti-VEGFR-1 antibody inhibited tumor growth; Lu et al. (2001) using a bifunctional antibody capable of binding to both VEGFR-1 and VEGFR-2 reported that this appeared to be a more effective inhibitor of VEGF signaling; and Zhu (2011) disclosed the production and utility of human anti-VEGFR-2 (KDR) antibodies as a means of inhibiting tumor growth. None of the prior art however disclose the use of anti-VEGFR antibody as a means of treating ARMD.

This invention discloses the use of anti-VEGFR antibodies to prepare immunoliposomes incorporating anti-inflammatory drugs in order to target VEGFR expressed on vascular cells in the retinal layer and to release the anti-inflammatory drug in the immediate vicinity of the proliferating vascular cells. There is thus a dual therapeutic effect upon ARMD. First, the anti-VEGFR antibody by binding to cellular VEGFR will prevent the binding of VEGF to its receptor and thereby block the signal for proliferation. At the same time the anti-inflammatory drug is released from the immunoliposome and will inhibit the local inflammatory response. We propose that this dual therapeutic action will enable us to effectively treat both the dry and wet forms of ARMD.

It is obvious to those of skill in the art that other nanosized drug delivery vehicles such as micelles, dendrimers, nanoemulsions and the like can be used in lieu of liposomes. The means for preparing micelles, dendrimers, nanoemulsions and the like are well known to those of skill in the art. Such methods are included by reference and are within the scope of this invention.

It is obvious to those of skill in the art that other compounds capable of binding to VEGFR can be used as binding agents in lieu of anti-VEGFR antibodies. For example, aptamers and binding peptides are different classes of compounds that mimic the binding ability of antibody.

In one embodiment of this invention an aptamer that targets VEGFR-1 and/or VEGFR-2 is used to coat a drug delivery vehicle such liposomes, micelles, dendrimers, nanoemulsions, or nanoparticles. Aptamers are small (i.e., 40 to 100 bases), synthetic single-stranded oligonucleotides (ssDNA or ssRNA) that can specifically recognize and bind to virtually any kind of target, including ions, whole cells, drugs, toxins, low-molecular-weight ligands, peptides, and proteins. Each aptamer has a unique configuration as a result of the composition of the nucleotide bases in the chain causing the molecule to fold in a particular manner. Because of their folded structure each aptamer will bind selectively to a particular ligand in a manner analogous to an antibody binding to its antigen. Aptamers are usually synthesized from combinatorial oligonucleotide libraries using in vitro selection methods such as the Systematic Evolution of Ligands by Exponential Enrichment (SELEX). This is a technique used for isolating functional synthetic nucleic acids by the in vitro screening of large, random libraries of oligonucleotides using an iterative process of adsorption, recovery, and amplification of the oligonucleotide sequences. The iterative process is carried out under increasingly stringent conditions to achieve an aptamer of high affinity for a particular target ligand (Gold L. et al. 1993). In order to improve stability against nucleases found in vivo the oligonucleotides may be modified to avoid nuclease attack. They may for example be synthesized as L-nucleotides instead of the natural D-nucleotides and thus avoid degradation from the natural nucleases.

The art is silent on the use of an anti-VEGFR aptamer as a targeting agent attached to the surface of liposomes and/or other drug delivery vehicles such as micelles, dendrimers, nanocapsules, nanoemulsion and nanoparticles, to deliver anti-inflammatory drugs to the immediate vicinity of the retinal layer of the eye.

It will also be obvious to those of skill in the art that another example of a binding agent that mimics the action of an antibody is a binding peptide. For example, a binding peptide that targets VEGFR can be attached to the surface of a liposomal drug and used to target VEGFR present on vascular cells. There are various methods for preparing synthetic or biological peptide libraries composed of up to a billion different sequences, and for identifying a particular peptide sequence that will target a particular protein such as VEGFR (Geysen H. M. et al. 1993; Zwick M. B. et al. 1998). The VEGFR binding peptide can be attached to a liposomal drug or other nanosized drug delivery vehicles using known methods and used to target the site of vascular proliferation.

The art is silent on the use of an anti-VEGFR binding peptide as a targeting agent attached to the surface of liposomes and/or other drug delivery vehicles such as micelles, dendrimers, nanocapsules, nanoemulsion and nanoparticles, to deliver anti-inflammatory drugs to the immediate vicinity of the retinal layer of the eye.

In this invention we disclose a means of treating ARMD using a variety of nanocarriers coated with an anti-VEGFR targeting agent. The VEGFR targeting nanocarrier is injected into the affected eye of the patient with ARMD into the immediate vicinity of the proliferating blood vessels supplying the retina. The anti-VEGFR targeting agent on the drug delivery vehicle will bind to the VEGFR on the vascular cells in the retina and inhibit their abnormal proliferation. At the same time the nanocarrier becomes bound to the vascular cells, and the anti-inflammatory drug is thereby released into the immediate vicinity of the retina where it will suppress the local inflammation. The combined therapeutic action of inhibiting abnormal proliferation of the neovascular cells as well as inhibiting the local inflammation may be an effective treatment for all forms of ARMD.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, said modifications are considered to lie within the spirit and scope of this invention.

### REFERENCES

Ellis et al. (2008) Nature Reviews Cancer 8, 579-591
Ferrara et al. (1999) Nat Med 5: 1359-1364.
Folkman (1971) N Engl J Med 285(21):1182-6.
Folkman (1995) Nat Med 1:27-31
Geysen H. M. et al. (1993). Bioorganic & Medicinal Chemistry Letters 3(3):397-404
Gold L. et al. (1993). U.S. Pat. No. 5,270,163
Hoeben et al. (2004) Pharmacol Rev 56:549-580
Huber et al (1999) Cancer Res 5:5209-5218
Jain K. (2005). Technology in Cancer Research and Treatment 4(4):407-416.
Kim et al. (1993) Nature (Iond.) 362:841-844.
Loges et al. (2010) Genes & Cancer 1(1):12-25
Lu et al. (2001) Cancer Res 61:7002-7008.
Luttun et al (2002) Nat med 8:831-840.
Park J. W. et al. (1997). Adv Pharmacol. 1997; 40:399-435
Park J. W. et al (2002). Clin Cancer Res. 2002; 8:1172-81.
Rockwell et al (2003) US Patent Application 20030108545
Schmidt J. et al. (2003) Brain 126:1895-1904
Torchilin V. P. AAPS Journal (2007), 9(2): E128-E147
Vasir J. K. et al (2005). Current Nanoscience 1:47-64.
Zhu Z. (2011) U.S. Pat. No. 8,057,791.
Zwick M. B. et al. (1998). Current Opinion in Biotechnology, 9(4):427-436

What is claimed is:

1. A method of treating both the “dry” and/or “wet” forms of Age Related Macular Degeneration (ARMD) by administering into the eye of the patient in need a therapeutic amount of a biopharmaceutical composed of an anti-inflammatory drug incorporated into a nanocarrier whose surface is coated with an anti-VEGFR targeting agent.

2. According to claim 1 the nanocarrier is either liposomes, or micelles, or dendrimers, or nanocapsules, or nanoparticles, or a nanoemulsion.

3. According to claim 1 the targeting agent is an anti-VEGFR antibody, or an anti-VEGFR aptamer, or an anti-VEGFR binding peptide.

4. According to claim 1 the anti-inflammatory drug includes corticosteroids, immune inhibiting drugs, and immune modulating drugs.

5. According to claim 1 a means of treating Age-Related Macular Degeneration (ARMD) by delivering said biopharmaceutical into the immediate vicinity of the retinal layer of the eye.
